(19) United States
(12) Patent Application Publication
(10) Pub. No.: US 2010/0174342 A1
(43) Pub. Date: Jul. 8, 2010

(54) TREMOR REDUCTION SYSTEMS SUITABLE FOR SELF-APPLICATION AND USE IN DISABLED PATIENTS

(76) Inventors: Leon Boston, Netanya (IL); Gal Ben-David, Adi (IL)

Correspondence Address: DEKEL PATENT LTD., DAVID KLEIN, BEIT HAROF'IM, 18 MENUHA VENAHALA STREET, ROOM 27, REHOVOT, 76209, IL

(21) Appl. No.: 12/513,667

(22) PCT Filed: Nov. 26, 2006
(86) PCT No.: PCT/IL06/01359

§ 371 Date: May 6, 2009

(51) Int. Cl. A61N 1/36

(52) U.S. Cl. 607/48

(57) ABSTRACT

Apparatus for tremor reduction including a sensor for sensing muscle movements, a stimulation/recording electrode unit for providing Functional Electrical Stimulation (FES) to a muscle, the stimulation/recording electrode unit including a filter for filtering around a tremor frequency to ignore slow movements and high frequency noise associated with the muscle movements that were sensed, and a processor for generating a set of relationships of muscle response to the FES, called FES-muscle-response relationships, and for selecting a new FES for application to the muscle in accordance with acquired knowledge of the FES-muscle-response relationships. An apparatus and a helmet for effecting proper alignment and application of a stimulation/recording electrode to a patient's arm and neck region, respectively, are also described.

### FIELD OF THE INVENTION

The present invention generally relates to a bio-electrical device and system. More specifically, the present invention relates to an apparatus, system and method for reducing deleterious involuntary tremors in the human body. The apparatus, system and method are especially configured to enable self application by a disabled patient, as well as to allow a physically impaired patient to independently prepare the device for repeated application and use.

### BACKGROUND OF THE INVENTION

Deleterious tremors and involuntary motions of various parts of the human body pose a considerable health problem and result in substantial loss of quality of life. The impairment of proper motor function resulting from such deleterious tremors and involuntary motions have far reaching consequences for the sufferer, which include interference with basic normal motor function which may be grossly incapacitating.

Both volitional and involuntary movement of any given body region are brought about by the action of muscle contraction and relaxation, which in all cases is endogenously neurally mediated. Inappropriately coordinated muscle activity results in the abnormal movements characterizing these disorders.

An artificial way of causing muscle contraction, which may effectively override the natural neuro-muscular mechanism, is by way of intra-muscular or body surface application of externally applied electrical stimulation to activate motor nerves.

This type of artificially induced muscle activity has been used to treat tremors and the like by the appropriately timed selective stimulation of muscles to counterbalance the undesirable activity, and thus eliminate the deleterious motion.

This method is most effective when used in combination with a means of providing feedback as to the state of motion of the body part being treated, and computer based control means for subsequently regulating stimulation so as to achieve the desired clinical effect.

If for example a subjects arm is being treated, then feedback of the arm's motion can be used to control the mode, magnitude and site of electrical stimulation, based on the feedback detection and control system. Likewise, endogenous neural activity, detectable by conventional means, can be used for providing feedback for applied electrical stimulation, either independently or in combination with motion as mentioned above.

The use of signals from switches or sensors to control functional electrical stimulation (FES) of paralyzed muscles is known. For example, U.S. Pat. No. 5,562,707 to Prochazka et al. describes many approaches in the prior art, such as use of a shoulder position sensor to control wrist extension via an implanted FES stimulator (Vodovnik, L. (1971) Development of Orthotic Systems using functional electrical stimulation and myoelectric control, Progress Report, University of Ljubljana, prepared for U.S. Dept. of Health Education and Welfare Social and Rehabilitation Service, under contract No. SRS-YUGO 23-68), shoulder sensors for controlling hand opening and pinch-grip (Peckham, P. H., Marsolais, E. B. & Mortimer, J. T. (1980) J. Hand Surgery, 5, 462-469; Peckham, P. H. & Keith, M. W. (1992) “Motor prostheses for restoration of upper extremity function” In: Neural Prostheses: Replacing Motor Function After Disease or Disability eds.: Stein, R. B., Peckham, P. H. & Popovic, D. B. New York: Oxford University Press; and U.S. Pat. No. 4,558,704 assigned to Wright State University, and a wrist position sensor for controlling FES of leg muscles (Prochazka, A. & Wiles, C. M. (1983) “Electrical stimulation of paretic leg muscles in man, allowing feedback-controlled movements to be generated from the wrist” J. Physiol. 343, 20P).

U.S. Pat. No. 5,562,707 itself describes a non-invasive self-contained functional electrical stimulation garment. The garment, which is preferably in the form of a glove, may be donned in one piece by a user of reduced motor ability, e.g., a person exhibiting hand tremors or who is a quadriplegic, paraplegic or hemiplegic. The garment is preferably made of a perforated elastic material. The garment has electrical connections internal to the garment that are adapted to make electrical contact with self-adhesive skin electrodes on the user.

However, a problem exists wherein the traditional prior art lacks a suitable method for enabling the handicapped patient to independently apply the treatment system in a sufficiently accurate way to allow the system to perform reliably and reproducibly, given the motor skill limitations which the tremor condition imposes.

U.S. Pat. No. 5,562,707, as mentioned above, utilizes a glove-like embodiment for applying the anti-tremor system to a patient, wherein multiple fixtures need to be engaged and then tightened in order to apply the device. The application of such an arrangement is further complicated due to the complex shape of the treatment device which requires correct initial placement even before the fixtures can be engaged and adjusted. Clearly, the practical usefulness of such a device for patients suffering from motor impairment is diminished in view of the relatively difficult application process required. Additionally, it would be problematic for a patient having involuntary motions and tremors to apply the device to themselves.

Another problem that exists in the prior art is the requirement for placement of the electrode-type device. Typically the patient has random tremors and involuntary bodily movements. The devices are most needed when the involuntary movements are most severe. The application of the electrode device to the needed body part can be inhibited because in order for the electrode device to accurately and effectively work, the device must be placed correctly. However, because of the involuntary movements, the patient often times is unable to correctly or accurately place the device on the needed site.

Further, even if the patient is able to properly fit the device on the site, typically the device will not work properly because of improper alignment with the muscle to be controlled.

A need therefore exists for a highly reliable and accurate method for applying an anti-tremor device. In addition, a need exists for an apparatus, system and method for applying and delivering an anti-tremor device by a patient suffering from involuntary tremors and wherein the apparatus, system and method allow for correct, reliable and accurate placement of the device on the desired tremor location.

Additionally, a need exists for an apparatus, system and method for applying an anti-tremor device in a manner which requires the least possible motor skill. Moreover, a need exists for an anti-tremor application with substantial improvements over the current state of the art, as will be described in detail below.

### SUMMARY OF THE INVENTION

The present invention seeks to provide an effective apparatus, system and method for reducing tremors at a location on the body by means of a closed-loop functional electrical stimulation device. The closed-loop functional electrical stimulation device may be applied in a highly reliable and accurate manner by the patient requiring reduction of the involuntary movements, yet require the least possible motor skill for independent self-application by handicapped patients. The invention is described for a single pair of muscles, but may be easily extended to a multiplicity of muscles.

The present invention seeks to provide apparatus for enabling independent, highly reliable and accurate self-application of an anti-tremor means by a disabled patient, without requirement for application fixtures, wherein anti-tremor means comprises a single unit, self adhesive stimulation and recording electrode with an integrated supply of energy and a control unit, further comprising an alignment, and application means for applying said anti-tremor means configured to enable independently self application by a severely handicapped patient, wherein system and method for tremor reduction is by means of closed-loop functional electrical stimulation, including a sensor for sensing muscle movements, and Functional Electrical Stimulation (FES) apparatus for providing FES to a muscle, the FES apparatus being in communication with the sensor via a band pass filter for filtering around a tremor frequency to ignore slow movements and high frequency noise.

There is thus provided in accordance with an embodiment of the present invention a method for tremor reduction including sensing muscle movements, providing Functional Electrical Stimulation (FES) to a muscle, including filtering around a tremor frequency to ignore slow movements and high frequency noise associated with the muscle movements that were sensed, generating a set of relationships of muscle response to the FES, called FES-muscle-response relationships, and selecting a new FES for application to the muscle in accordance with acquired knowledge of the FES-muscle-response relationships.

The new FES may be selected so as to optimize tremor reduction. The new FES may be selected by on-line calculation of a correlation between FES stimulation and actual muscle movements.

The method may include sensing further muscle movements, comparing the further muscle movements with previously stored FES-muscle-response relationships, selecting the new FES in accordance with the previously stored FES-muscle-response relationships wherein the new FES may be selected so as to oppose unwanted vibration of the muscle, and applying the new FES to the muscle.

The FES-muscle-response relationships may be modified in response to physiological changes of the muscle being stimulated.

The muscle movements may be sensed by an accelerometer, e.g., a MEMS (micro-electro-mechanical system) accelerometer.

There is also provided in accordance with an embodiment of the present invention apparatus for tremor reduction including a sensor for sensing muscle movements, a stimulation/recording electrode unit for providing Functional Electrical Stimulation (FES) to a muscle, the stimulation/recording electrode unit including a filter for filtering around a tremor frequency to ignore slow movements and high frequency noise associated with the muscle movements that were sensed, and a processor for generating a set of relationships of muscle response to the FES, called FES-muscle-response relationships, and for selecting a new FES for application to the muscle in accordance with acquired knowledge of the FES-muscle-response relationships.

The stimulation/recording electrode unit may include a first layer including an adhesive and conductive interface layer adapted to directly interface with a skin surface and to provide electrical contact for conducting electrical energy to the skin surface for effecting muscle contraction and for detecting bio-potential signals indicative of neuro-muscular activation, a second layer including a flexible conducting layer adapted to provide electrical contact between the skin surface and a source of electromotive force (EMF) and to electrically connect the skin surface to a control unit for electrical stimulation of the skin surface, and a third layer including an outer flexible layer that includes a source of electrical energy and serves as a protective covering layer, the third layer including a portal for installing therein the control unit.

The apparatus may further include an arm alignment and electrode application unit that includes a pathway for insertion of a patient's arm therethrough, the pathway having a limited width so that the patient's arm with fingers outstretched is forced to pass through the pathway parallel to a vertical axis of the pathway.

The stimulation/recording electrode unit may include interface magnets and the arm alignment and electrode application unit may include interface magnets corresponding to a position of the interface magnets of the stimulation/recording electrode unit but of opposing polarity, to prevent inverse placement of the stimulation/recording electrode unit on the patient's arm.

### BRIEF DESCRIPTION OF THE DRAWINGS

Various objects, features, aspects and advantages of the present invention will become more apparent from the following detailed description of preferred embodiments of the invention, along with the accompanying drawings in which like numerals represent like components

FIGS. 1A-1C are simplified illustrations of a stimulation and/or recording electrode, in accordance with an embodiment of the present invention.

FIG. 2 is a simplified illustration of an apparatus for effecting the proper alignment and application of a stimulation and/or recording electrode, in a minimally user dependent manner, in accordance with an embodiment of the present invention.

FIGS. 2A-2E are simplified illustrations of a system for tremor reduction by means of closed-loop functional electrical stimulation, for use as a helmet, in accordance with an embodiment of the present invention.

FIG. 3 is a simplified illustration of a system for tremor reduction by means of closed-loop functional electrical stimulation, in accordance with an embodiment of the present invention.

FIG. 4 is an exemplary graph of muscle movement as a function of Functional Electrical Stimulation (FES). The graph is an exemplary outcome of the adaptive calibration process as described further below (methods or algorithms for adaptive stabilization for tremor, in accordance with embodiments of the present invention) with reference to FIG. 7.

FIG. 5 is a simplified block diagram of a stabilization filter useful in the system of FIG. 1, in accordance with an embodiment of the present invention.

FIG. 6 is a simplified illustration of a dynamic model of an algorithm input, used in methods for adaptive stabilization for tremor, in accordance with embodiments of the present invention, e.g., in measuring single axis tremor using a 2-D accelerometer.

FIG. 7 is a simplified illustration of a tremor reduction system, in accordance with an embodiment of the present invention, which activates two muscles that apply up and down force.

### DETAILED DESCRIPTION OF THE EXEMPLARY EMBODIMENTS

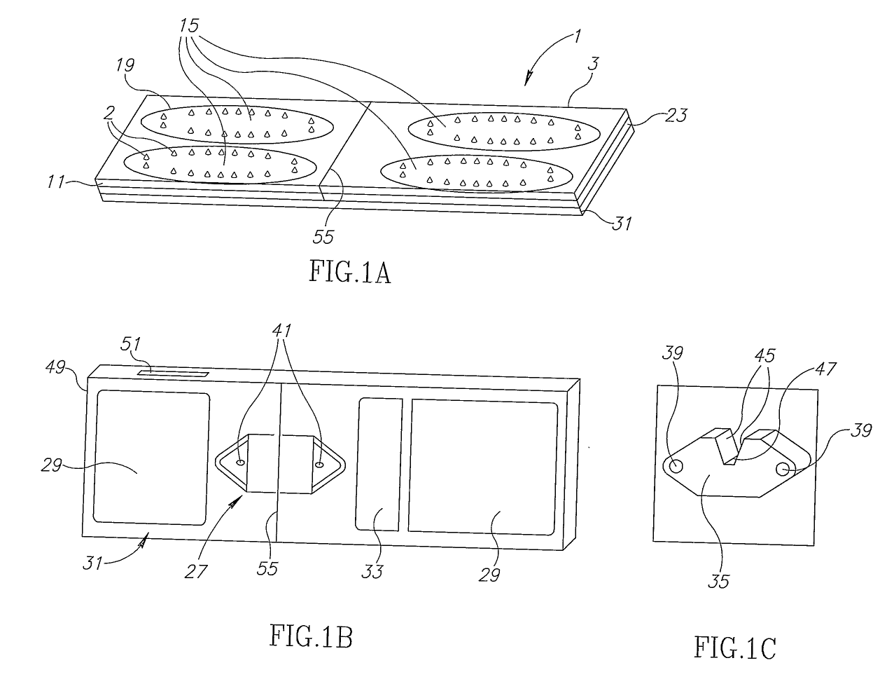

Reference is now made to FIGS. 1A-1C, which illustrate the stimulation/recording electrode unit 1, in accordance with an embodiment of the present invention.

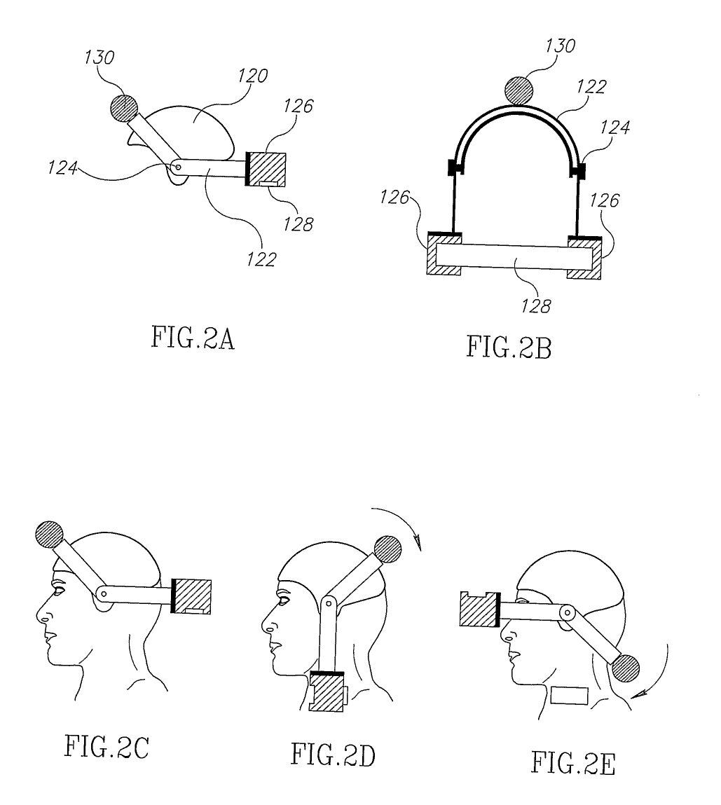

Unit 1 may include, without limitation, three thin layers. The first layer is an adhesive and conductive interface layer 3 which may directly interface with the skin surface of a patient. The first adhesive and conductive interface layer 3 may provide the electrical contact necessary (e.g., via electrodes 2) for the functions of conducting electrical energy to the skin surface for effecting the muscle contraction (illustrated in FIG. 3), and for the detection of naturally occurring bio-potential signals such as the electro-myographic (EMG) signals indicative of neuro-muscular electrical activity.

The adhesive and conductive interface layer 3 in an exemplary embodiment may include a hydrophilic “hydrogel” material 11 of which there are many commercially available varieties.

The first (hydrogel) layer 3 may possess strong adhesive properties as well as high electrical conductivity to facilitate the acquisition of bio-electrical signals from the body surface as well as the application of the electrical stimulation.

The first (hydrogel) layer 3 may be subdivided into electrically discrete regions 15 by creating gaps in the first (hydrogel) layer 3 so as to generate vacant perimeter borders 19 circumscribing electrically isolated regions 15, for the purpose of allowing selective stimulation and/or recording.

A second flexible conducting layer 23 may be provided wherein the second flexible conducting layer 23 may channel the flow of electrons between the first layer 3 and the device's control system 27 (such as with contacts or vias), described in greater detail below. The flexible conducting layer 23 may provide electrical contact between the first layer 3 and a source of electromotive force (power source). Additionally, the flexible conducting layer 23 may provide a means for acquiring the bio-electrical signals. Moreover, the flexible conducting layer 23 may provide a means for connecting the skin contacting surface to the control system 27 so as to allow appropriate electrical stimulation or recording of the bio-potential signals as determined by the control system 27. The flexible conducting layer 23 may be comprised, for example, of electrically conductive rubber, single or double sided flexible printed circuit board material or any other suitable electrical conducting means which possesses an appropriate degree of flexibility.

The third outer flexible layer 31 may be provided on the stimulation recording electrode unit 1. The flexible outer layer 31 may serve as a source of electrical energy and also may serve as a protective covering layer. In an exemplary embodiment, a power source 33 may be contained thereon in the outer flexible layer 31. Many different types of power sources 33 may be utilized to power the electrode unit 1. In an exemplary embodiment of the present invention, so called “paper batteries” may be utilized to power the electrode unit 1. The outer flexible layer 31 may also serve as a portal for the placement of a re-usable control unit 27 (FIG. 1C) which regulates the feedback controlled stimulatory activity as well as integrating the input information, such as the motion characteristics, which will be described in greater detail below.

As further illustrated in the lower panel (FIG. 1C), a control unit (or insertion) portal 35 for the reusable control unit 27 is shown. FIG. 1B illustrates an exemplary embodiment of control unit 27 inserted in the respective outer flexible layer 3. As described above, the outer flexible layer 3 may serve a reservoir of electrical energy and more specifically of the power source 33. Moreover, the outer flexible layer 31 may conduct with the flexible conducting layer 23 (such as with contacts or vias) which interfaces between the stimulating and/or data acquiring electrodes 2 and the control unit 27. It should be noted that the control unit portal 35 is designed to simplify insertion and orientation of the control unit 27, by virtue of a number of features including: a pair of magnets 39 for receiving magnets 41 of opposite polarity at corresponding positions on the control unit 27, thus ensuring that inverse placement of the control unit 27 is impossible, and further ensuring that the control unit 27 is properly oriented with respect to the electrode unit 1. The control unit portal 35 may have an insertion portal 47 with conical side walls 45, which mate with a complementary shaped slope of the control unit 27, to facilitate the smooth alignment of the control unit 27 with respect to the electrode unit 1 and to ensure accurate placement of the control unit 27 with respect to the electrode unit 1.

Collectively, these features allow the control unit 27 to be placed roughly in the region of the insertion portion since the magnets 39 (co-acting with magnets 41) will then complete the positioning process and fix the control unit 27 accurately in place, such that the control unit 27 may be easily slid into the insertion portal 47 even in the presence of severe body tremors by the patient.

In an exemplary embodiment, a relatively large region, or even the entire external surface 49 of the multi-layer unit 1 may be configured to function as an on/off switch 51. This on/off switch 51 may be activated or deactivated by the patient simply pressing any portion of the external surface 49 of the electrode unit 1. In another exemplary embodiment, the electrode unit 1 may activated or deactivated by using a voice activated means (not shown) known in the art.

It is advantageous in the present invention to allow for removal of the multi-layer electrode unit 1 and allow for extraction of the re-usable control unit 27. In an exemplary embodiment of the present invention, the electrode unit 1 may be easily self-applied by a disabled patient (not shown), so too is it vital that it be equally easily removed. Although it would be relatively easy for many patients to simply pull the device 1 off, for those unable to do so it would be possible to place the application site under a steady and sustained stream of water. Since the adhesive layer 3 is strongly hydrophilic, it will after time become water logged and lose its adhesiveness to the extent to which the stream of water can then dislodge it.

Extracting the re-usable control unit 27 can be easily achieved by simply folding the multi-layer electrode unit 1 along a prepared fault line 55 running through the midline of the control unit 27. Since the opposing sides of the hydrogel surface 3 retain a degree of adhesiveness after removal (even after the above described stream of water method), the act of folding the unit 1 along the fault line 55 will result in a bending moment strong enough to disrupt the magnetic contact which held the control unit 27 to the multi-layer unit 1.

The above described multi-layer electrode unit 1 may be characterized as having a high degree of flexibility to allow it to conform to the contour of the particular body surface region to which it is to be applied. The multi-layer electrode unit 1 may also be further characterized as being highly adhesive over its entire body contacting surface so that it readily adheres to the body surface and remains firmly attached to the intended site of application. Further features of the multi-layer electrode unit 1 may include a means for easily switching the unit 1 on or off, as well as simple means for removing the unit 1 and extracting the re-usable control unit 27.

The multi-layer electrode unit 1 in an exemplary embodiment may have a special arrangement of side mounted magnets 29 of like polarity, as shown in the lower panel of FIG. 1, for facilitating proper orientation of this unit 1 with respect to the opposite polarity at corresponding positions on an arm alignment and electrode application unit 63. The two units have corresponding magnets 29 of opposing polarity, to ensure that inverse placement of the multilayer complex is impossible, and further ensuring that it is properly oriented with respect to the arm alignment and electrode application unit 1. Conical, or fluted side wall design of the insertion region of the arm alignment and electrode application unit 1, and inverse slope of the multi-layer unit, further facilitate smooth alignment between the two units.

Alternative methods for non-permanently fixing the multi-layer unit to the arm alignment and electrode application unit include surface adhesive attachment or multiple hook fasteners (“Velcro” strips).

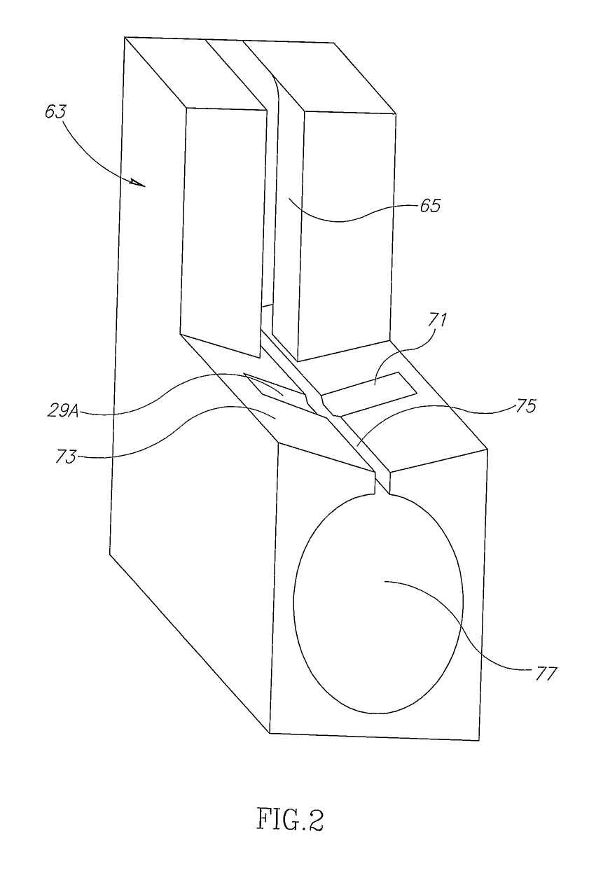

FIG. 2 illustrates an apparatus 63 for effecting the proper alignment and application of a multi-layer stimulation and/or recording electrode 1 of the type described above, in an essentially user independent manner. For purposes of explanation the apparatus 63 is described for application to a patient's arm, however it should be understood that application to other body sites is also possible.

The arm alignment and electrode application unit 63 may be intended to accurately and non-permanently tether the multi-layer stimulation and/or recording electrode unit 1, and to further set the correct arm orientation, so that the multi-layer unit 1 becomes correctly attached to the patient's body surface to permit accurate and effective anti-tremor treatment.

As can be seen in FIG. 2 (for brevity, only the left hand side image is labeled, the right hand side image having corresponding structures), there is a narrow pathway 65 for the vertical insertion of the patients hand (not shown). The limited width of this region ensures that the patient must position the hand with fingers outstretched and position the hand parallel to the axis of the vertical insertion pathway 65. This may ensure the correct orientation of the arm.

Another feature of the arm alignment and electrode application unit 63 is a special niche 71 for receiving the multi-layer unit 1. This niche 71 possesses magnets 29A corresponding to the position of the magnets 29 in the multi-layer unit 1 but of opposing polarity, to ensure that inverse placement of the multilayer complex is impossible, and to further ensuring that it is properly oriented with respect to the arm alignment and electrode application unit 1.

A conical, or fluted side wall 73 design of the insertion region of the arm alignment and electrode application unit 63, and inverse slope of the multi-layer unit 1, further facilitate smooth alignment between the two units for easy application by a handicapped patient. In practice, the multi-layer unit 1 needs only to be roughly placed in the region of niche 71 since the magnets 29 will then complete the positioning process and fix the multi-layer unit 1 accurately to the arm alignment and electrode application unit.

The correct distance between the finger tips and the multi-layer unit on the patient's arm is determined by virtue of the distance between the insertion niche 71 and the side wall 73 being tailored to the patient's arm size.

After the arm has passed through the insertion pathway 65, it will come into contact with the adhesive surface 3 of the previously placed multi-layer unit 1, placed at the insertion niche 71. The magnetic force holding the opposing ends of the multi-layer unit 1 in place may provide sufficient resistance to cause the multi layer unit 1 to become properly adhered to the patient's skin before it can become dislodged from the magnetic binding sites.

With further application of downward force, the patient's arm, now with the multi-layer unit 1 at least partially attached, comes into contact with the upper side sponge walls 75 (alternatively formed of foam or other suitable compliant and elastic material), which apply even pressure to the electrode attaching it to skin surface as the arm is forces between the two sides and finally brought all the way into the sponge walled cylindrical cavity 77, of the arm alignment and electrode application unit 1. The cavity 77 is configured to snugly fit around the arms perimeter to apply gentle force to further ensure complete attachment of the multi-layer unit 1 to the patient.

Having finally brought the arm into cavity 77, the multi-layer unit 1 will have been completely attached to the treatment site, and thus the arm may be withdrawn from the unit 77 with the multi-layer unit 1 firmly in place in the optimal region for effecting the feedback controlled anti-tremor function soon to be described.

As can be appreciated from the above description, the combined use of the multi-layer unit 1 and the arm alignment and electrode application unit 63 facilitates the application of the anti-tremor means in a consistent manner, to ensure correct orientation and location. Furthermore, the foam housing may provide support for trembling arm, obviating the need for the use of the second hand and causing minimal interference with blood circulation during the application process.

Thus, the application of the multi-layer unit 1 merely requires the patient to place the hand in the pathway 65, push the arm down and then remove the arm.

As can be understood, the above described multi-layer units 1 and arm alignment and electrode application units are effective for providing feedback controlled electrical stimulation to counteract abnormal tremor activity.

The successful performance of this goal is critically dependent on the accurate placement of the stimulation electrodes 1 in correct proximity to the appropriate muscles.

The following four parameters define the appropriate configuration of the multi-layer stimulation/recording unit for effecting appropriate treatment at differing body locations:

1. anatomical variations from body region to body region,

2. the side of the body being treated (left or right), and

3. size variation between patients.

The problem may be dealt with in the following ways:

1. Anatomical variations between differing body regions may be dealt with by appropriately configuring the partitioning of stimulation/recording regions of the multi-layer unit 1 in accordance with the specific myology of the respective treatment sites, knowledge of which is well known to the medical literature.

2. Similarly, the sidedness issue can be resolved by appropriately configuring the partitioning of the multi-layer unit 1 in accordance with the appropriate myology of the respective treatment sites.

3. Size variation between patients can be addressed by appropriately scaling the multi-layer unit 1 in accordance with the girth of the respective treatment sites (i.e., having various sized units to accommodate various size ranges), and scaling the alignment and electrode application unit in accordance with the length of the respective treatment sites by placing the insertion niche of the alignment and electrode application unit 63 an appropriate distance from the terminal extremity of the alignment and electrode application unit (i.e., having various lengths between these points), so that the multi-layer unit 1 is placed at the optimal position along the length of the given treatment site.

To properly effect the correct placement of multi-layer units 1 at various other sites of the body, certain variations in the design of the alignment and electrode application unit 63 may be required. An illustrative case in point is in the placement of multi-layer units 1 to the neck region, as described below.

While it may be possible to use an alignment and electrode application unit of the general form of 63, which has no moving parts, but which is constructed to accommodate a patient's head, it may however be advantageous to mount the electrode application unit into a system which allows the application unit to move with respect to the patient's body, rather than the reverse.

For example, applying the alignment and electrode application unit onto a movable mounting attached to a helmet may allow the multi-layer unit (1) to be swiveled into place so as to be correctly attached to the patient's body surface to permit accurate and effective anti-tremor treatment.

Reference is now made to FIGS. 2A-2E, which illustrate a system for effecting proper alignment and application of a stimulation and/or recording electrode, utilizing a helmet, in accordance with an embodiment of the present invention. The system includes a helmet 120 with a pivoted arm 122 that is mounted for rotation about a pivot 124. An electrode application unit 126 (similar to the unit 63 described above) may be mounted on pivoted arm 122. For example, the two sides of the electrode application unit 126 may be mounted on opposite sides of pivoted arm 122, connected by a connecting member 128. The multi-layer unit may be placed into the niches formed in the walls of the electrode application unit 126 as previously described. Once the multi-layer unit is in place, the helmet 120 may be positioned on the patient's head, after which pivoted arm 122 may be rotated with the aid of a knob 130, so as to adhesively apply the multi-layer unit to the patient's neck region as illustrated in FIGS. 2C-2E. Following application, the application device can be removed. The multi-layer unit will thus be correctly positioned for providing optimal stimulation and tremor detection without having required any exacting coordinated activity on the part of the patient or any person performing the application.

Individual variations in the distance between the optimal treatment site and the position of the helmet on the skull can be readily adjusted for by appropriately setting the relative position of the alignment and electrode application unit components along the pivoted arm 122 and/or connecting member 128. Any method known to the art for adjustably setting the relative position would be suitable.

Reference is now made to FIG. 3, which illustrates a system for tremor reduction by means of closed-loop functional electrical stimulation, in accordance with an embodiment of the present invention.

The muscles of the upper arm 83 are an example of dual action muscles, antagonistic muscles, including a flexor 85, a muscle that bends a joint 87, and an extensor 89, a muscle that straightens a joint 87. When the biceps muscle 91 (on the front of the upper arm, flexor 85) contracts, it bends or flexes the elbow joint 87. When the triceps muscle 93 (on the back of the upper arm, extensor 89) contracts, it opens, or extends, the elbow joint 87.

A controlled movement requires contraction by both muscles 91, 93. A muscle pulls when it contracts, but exerts no force when it relaxes and cannot push. When one muscle pulls a bone in one direction, another muscle is needed to pull the bone in the other direction.

A normal characteristic of all skeletal muscles is that they remain in a state of partial contraction. At any given time, some fraction of the muscle's contractile elements (myocytes or muscle fibers) are being stimulated while others are not. This causes a partially tightened, or flexed, muscle and is known as muscle tone, the extent of which can vary over time.

The closed loop tremor reduction system senses the overall movement of the arm that is composed of an intentional movement and tremor. Distinguishing between intentional movement and tremor is impossible unless one can guess what the patient's intentions are. Therefore we differentiate between “intentional” low frequency signals (around <1 Hz) and high frequency (around >1 Hz) signals. In general, the system ignores low frequency movements and restrains high frequency movements. One should note that the earth's gravity g—is a constant “low frequency” factor that may be ignored.

The restraint of high frequency movement is made by generating Functional Electrical Stimulation (FES) at an appropriate body surface location to activate the muscle that generates an opposite action.

An accelerometer 94 may be used to measure the acceleration of the arm or other body region with electronic equipment 95. Most accelerometers 94 available for the measurements in biomechanics are extremely light and weigh only a few grams. There are various types of accelerometers 94, including but not limited to, MEMS (micro-electro-mechanical system), piezoresistive, strain gauge, piezoelectric, and inductive transducers. In a prototype of the invention, two ADXL202 low cost 2 g dual axis MEMS Accelerometers 94 may be used for three dimensional acceleration measurements. The ADXL202 allows bandwidth of about 50 Hz. (ADXL202 is manufactured by Analog Devices of One Technology Way, Norwood, Mass. 02062-9106.)

As is known in the art of measurement, there may be a small error in the measured acceleration, which is now discussed. The magnitude of the acceleration measured with an accelerometer 94 depends on various factors, e.g., bone acceleration, mounting interaction, angular motion and gravity. Accelerometers 94 are mounted by placement in the multi-layer unit described above, strapping, or otherwise attaching them to the segment of interest at a location with minimal soft tissue between the accelerometer 94 and the bone of interest. In any mounting case, the acceleration measured represents not “the bone acceleration” but rather the acceleration of a specific mass element at the surface of the bone or even of a point outside the bone. Acceleration of a specific bone location can then be determined mathematically from several acceleration measurements or from additional measurements.

Acceleration measurements on a segment of the human body provide a signal which is composed of translational, rotational, and gravitational components. Accelerometer signals measured during human or animal locomotion have different combinations of the three acceleration components, depending on the actual movement.

Functional Electrical Stimulation (FES) is a means of producing useful movement in muscles and is well known in the art. Electrical impulses are applied using either skin surface or implanted electrodes and cause muscles to contract in a controlled manner. Applications are found in spinal cord injury, stroke, MS and cerebral palsy to assist standing, walking and hand function. An important use for FES is as a means of producing useful movement in paralyzed muscles. The tremor reduction system, however, activates “free-run” rather than paralyzed muscles.

An electromyogram (EMG) represents the aggregate electrical activity produced by multiple action potentials that are generated by contracting muscle fibers. The EMG is not a regular series of waves like the ECG but rather a chaotic burst of overlapping high frequency signals (around 1 KHz) that are recorded using non-invasive surface electrodes 97. These bioelectrical signals are typically very small in amplitude (microvolts) and an amplifier is required to accurately record, display and analyze the EMG.

The above described motion-sensing apparatus and the system for tremor reduction by means of closed-loop functional electrical stimulation as illustrated in FIG. 3, may be integrated into a miniaturized form and be applied as control unit 27 of FIG. 1, in accordance with an embodiment of the present invention.

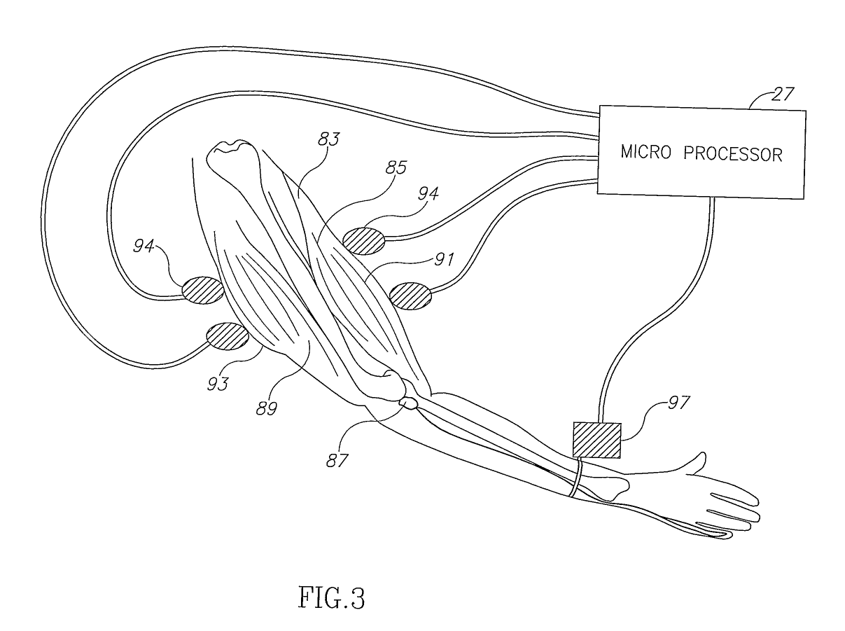

FIG. 4 further illustrates an exemplary graph of muscle movement as a function of FES. A stabilization algorithm may be used in the invention. The stabilization requires two concurrent tasks: calibration and filtering. The system may be manually attached to the muscles using self adhesive electrodes and the accelerometers may be an integral part of the control unit when using self adhesive electrodes units as described above, or may be attached using rubber bands (or Velcro bands or other means). Due to the diverse possibilities of attachments the effect of the FES on hand movement should be calibrated.

Calibration may be performed in the beginning of device activation (initial calibration) and at run time (fine tuning). Calibration may be performed by collecting FES stimulation parameters such as amplitude or other parameters of FES and movement response. Due to tremor, fluctuations are present at the movement axis. During initial calibration the statistics bank is built using full range tests initialized by the microprocessor 103. During run time the calibration is slowly tuned to reflect small changes in electrode conductivity, accelerometer movements, etc. The FES stimulus is limited for patient safety.

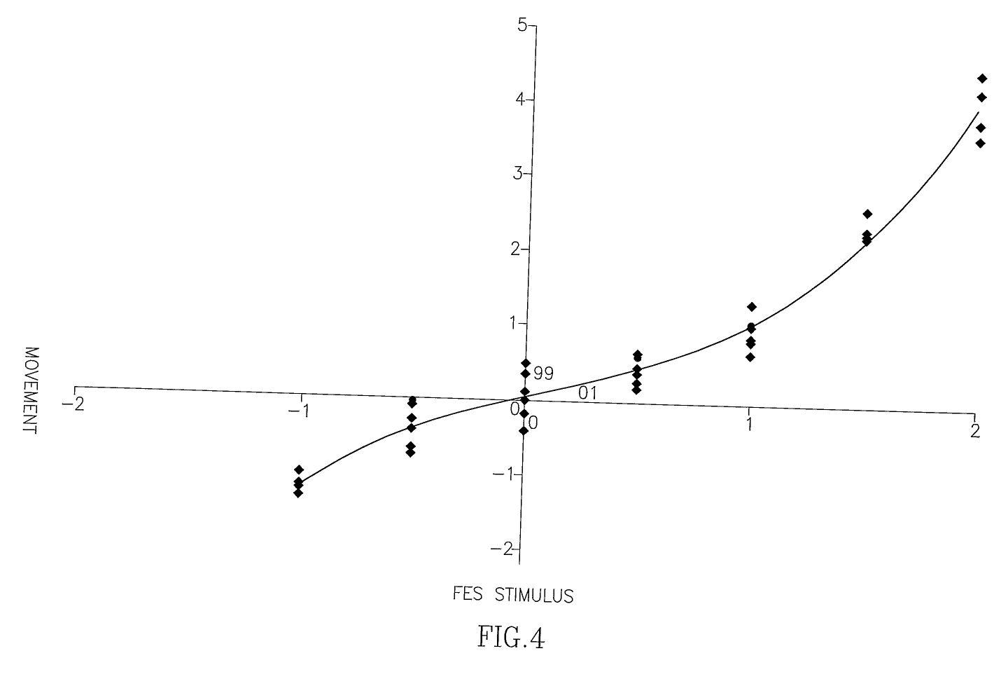

FIG. 5 illustrates a simplified block diagram of a stabilization filter useful in the system of FIG. 1, in accordance with an embodiment of the present invention. The stabilization filter may include the EMG 97, whose output is analyzed by a tremor frequency detection module 109. The accelerometer 94 outputs to a band pass filter 111. Movements are measured by the accelerometer 94 and muscle electric activity is measured by the EMG 97. Tremor frequency is detected from EMG 97 signal using peak FFT detection. Movements 113 are band pass filtered through band pass filter 111 around the tremor frequency (usually, but not necessarily, to 130 Hz) to ignore slow (“intentional”) movements and high frequency noise.

The desired movement is opposite to the sensed movement and the corresponding stimulus at the FES electrodes 121 is calculated using a calibration algorithm 119 (see below). Again, FES magnitude may be limited for safety. It is to be noted that FES amplitude may be considered to be any one or any combination of the well known stimulation parameters including but not limited to voltage, amperage, current density, pulse duration, spike duration, duty cycle, stimulation pulse waveform (square, sinusoidal, saw-tooth etc.), pulse train pattern (monophasic, biphasic, symmetrical, asymmetrical etc.) and pulse train pattern modulation (ramping, oscillating etc.).

The following is an explanation of methods or algorithms for adaptive stabilization for tremor, in accordance with embodiments of the present invention. This is a simplified description of the principle of the adaptive control as implemented in the wrist device embodiment of the invention, designed to sense and reduce tremor. The description is focused on a single axis (“up down”) movement for clarity.

Measuring Single Axis Tremor Using 2-D Accelerometer

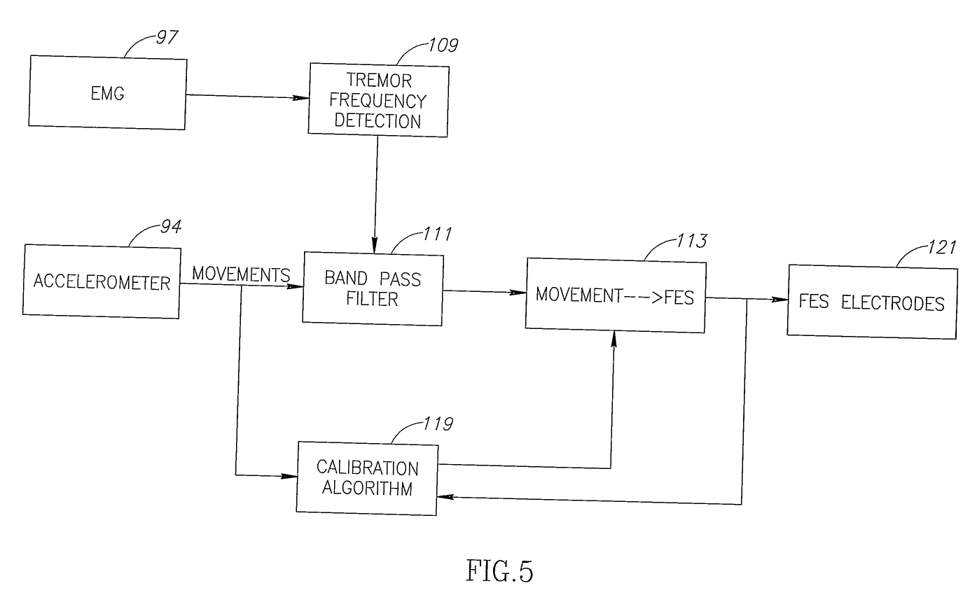

FIG. 6 illustrates the dynamic model of the algorithm input.

A dual axis accelerometer is placed on the back of patient's palm. The accelerometer senses accelerations that are either parallel (p) or orthogonal (o) to the surface. When the hand moves up and down, two acceleration components are sensed. The first is the gravity that present a constant force directed toward the center of the earth. The second acceleration component is due to the movements of the palm (tremor). For simplicity, one can assume that only up-down movements are present.

$$p ( t ) = g sin θ ( t ) \\ o ( t ) = - g cos θ ( t ) + r \frac{{d}^{2} θ ( t )}{d {t}^{2}} \qquad ( 1 )$$

where t represents time.

We are interested in obtaining the second time derivative of the angle θ (due to human limitations we may assume that it is limited to +/−45 degrees).

$$\begin{matrix}θ ( t ) = arc sin \frac{p ( t )}{g} & ( 2 ) \\ {A}_{a} ( t ) = \frac{{d}^{2} θ}{d {t}^{2}} = \frac{1}{r} [ o ( t ) + g cos ( arc sin \frac{p ( t )}{g} ) ] & ( 3 )\end{matrix}$$

It should be noted that the placement of the accelerometer changes the measurement of the angular acceleration by the factor r. This factor is mentioned later in the adaptation algorithm.

The angular acceleration is sampled at a sampling interval T that is chosen to comply with the Nyquist rule. In practice, sampling rate of 100-500 Hz was found to be adequate. The subscripts a and d are used to differentiate between analog and digital signals respectively.

A_{d}(n)=Aₐ(nT) (4)

Estimation of Band Limited Hand Velocity

The angular velocity of the palm is estimated by “integrating” the angular acceleration.

V_{d}(n)=α₁·V_{d}(n−1)+T·A_{d}(n) (5)

The angle is estimated by “integrating” the angular acceleration.

F_{d}(n)=α₂·F_{d}(n−1)+V_{d}(n) (6)

In its nature, integration has an infinite memory as it sums the measured acceleration since the device was turned on. Any error in measurement due to noise or mechanical impact will affect the system for a very long time. In order to decay such errors, a decay factor α₁,α₂ is added to equations 5 and 6. A typical value of α₁,α₂ is 0.99 to 0.999.

The angle F_{d}(n) is assumed to have three major frequency components. Low frequencies, typically up to 2 Hz are considered as “voluntary” movement and should not be affected by the device. Higher frequency movements, in the region 2-20 Hz are considered as tremor and should be stabilized. Signal frequencies above 20 Hz (typically) are assumed to be electronic and mechanical noise.

The location signal is therefore filtered, using a digital IIR 2-20 Hz band-pass filter. Note that the AC coupling nature of the filter assures that when no tremor movements are measured, no correction will be made. The output of the filter is denoted by I_{d}(n).

Muscle Activation

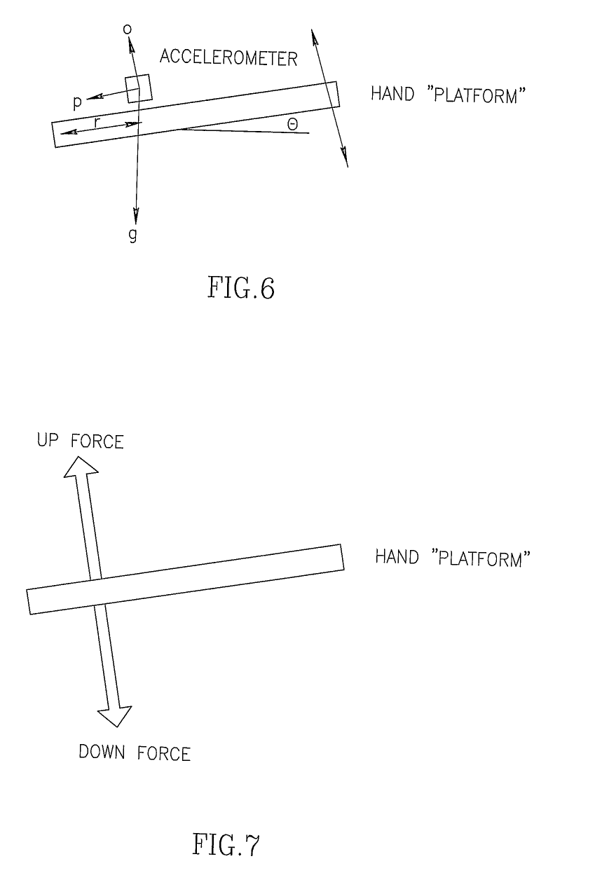

The tremor reduction system activates two muscles that apply up and down force as shown in FIG. 7.

Each muscle is activated by a separate FES circuit. FES circuits are known in the art and they generate pulses at few tens to few hundred Hertz. These pulses are modulated by two control signals, denoted U_{d}(n),D_{d}(n) respectively. (“Modulated” refers to, without limitation, amplitude modulated, e.g., voltage or current modulated, as well as being modulated by stimulation parameters other than amplitude, e.g., frequency, duty-cycle, and the like.) As the amplitude of U_{d}(n),D_{d}(n) get higher, the corresponding muscle is affected to generate stronger contraction. Since there is no “negative” contraction both envelopes are positive U_{d}(n),D_{d}(n)≧0

It should be emphasized that the correlation between the control signals U_{d}(n),D_{d}(n) and the actual organ movement is not known in advance. The conductivity of the electrodes and skin as well as muscle response may change among persons and even among placements on the same patient.

To summarize, the stabilizer's input is a band-pass filtered estimation of the palm angular location I_{d}(n). Its outputs are two positive control signals U_{d}(n),D_{d}(n)≧0 that set the amplitude of the FES pulses thus changing the stimulus of the up and down muscles.

Control Loop

The control loop goal is to activate force as a response to angular movements, but in the opposite direction. Movements up are corrected by form down and vice versa.

$${C}_{d} ( n ) = - {I}_{d} ( n ) \\ {U}_{d} ( n ) = \{ \begin{matrix}{β}_{1} ( n ) {C}_{d} ( n ) & {C}_{d} ( n ) \geq 0 \\ 0 & {C}_{d} ( n ) < 0\end{matrix} \\ {D}_{d} ( n ) = \{ \begin{matrix}{β}_{2} ( n ) {C}_{d} ( n ) & {C}_{d} ( n ) \leq 0 \\ 0 & {C}_{d} ( n ) > 0\end{matrix} \qquad ( 7 )$$

The mutually exclusive regions avoid a situation of positive feedback between the two antagonistic muscles.

The parameters β₁(n),β₂(n) are slowly varying and needed to compensate for the ratio between the electronic stimulation and muscle movements. The ratio is a function of accelerometer placement (parameter r), electrode placement and other for individual parameters. Both parameters are adapted to reduce the tremor to a minimum.

The previous work mentioned above by Arthur Prochazka tested fixed stabilization filters and could not compensate for differences among patients and electrode placement. The adaptation method allows the wrist device to “study” the characteristics of the hand/electrodes and stabilize accordingly.

Define positive and negative tremor energy as the tremor energy during the last N samples (p stands for positive and n for negative):

$${S}^{P} ( n ) = {\sum }_{n = 0}^{N - 1} {[ max \{ {C}_{d} ( n ) , 0 \} ]}^{2} \\ {S}^{N} ( n ) = {\sum }_{n = 0}^{N - 1} {[ min \{ {C}_{d} ( n ) , 0 \} ]}^{2} \qquad ( 8 )$$

The parameters β₁(n),β₂(n) are adapted by “tuning” their value to minimize both positive and negative tremor energy.

The tremor reduction is done by changing parameters β₁(n),β₂(n) to lower their corresponding tremor energy. For example, if β₁(n) was increased and S^{P}(12) is smaller, β₁(n) will be increased further, but if S^{P}(n) is larger, β₁(n) will be decreased. This method is known in the Signal Processing art as Steepest Descent (SD) or Least Mean Square (LMS) adaptation.

$${β}_{1} ( n ) = {β}_{1} ( n - 1 ) - Δ \cdot \frac{{S}^{P} ( n - 1 ) - {S}^{P} ( n - 2 )}{{β}_{1} ( n - 1 ) - {β}_{1} ( n - 2 )} \\ {β}_{2} ( n ) = {β}_{2} ( n - 1 ) - Δ \cdot \frac{{S}^{N} ( n - 1 ) - {S}^{N} ( n - 2 )}{{β}_{2} ( n - 1 ) - {β}_{2} ( n - 2 )} \qquad ( 9 )$$

Selection of the parameter Δ is a compromise between adaptation speed and stability. The theory of its selection known in the LMS art.

Small value of the parameter Δ will result in small adaptation steps. Such adaptation may require a long time to be effective. On the other hand when stabilized, the outcome of small adaptation steps is a relatively fixed stabilization filter. Large values of the parameter Δ result in faster adaptation, but after the “study” period, the stabilization filter has worse stabilization effect.

It is noted that in order to minimize vibrations, the amplitude of the antagonistic muscle response to the FES should be as close as possible the original tremor, yet in opposite direction. There is no direct or a-priori knowledge of the relation between the FES amplitude and the muscle response. The FES stimulus to muscle response relations depends on many factors. Some factors are personal parameters of the patient (muscle strength, non muscle tissues, and skin resistance), while other factors may be related to system configuration or human interface factors, like the placement of the electrodes. Over time, other factors like sweat and muscle fatigue may change these relationships. Therefore, some sort of tuning is needed in order to guarantee good stabilization over time.

The prior art (e.g., Prochazka et al.) uses off line tuning of system parameters. Such an approach may work in a laboratory environment where an engineer changes parameters in real time but is not appropriate for real life use, and therefore cannot be used in a commercial product.

The algorithm described above is adaptive in nature. It uses an automatic studying approach to learn the FES-stimulus to muscle-response relationship so as to optimize tremor reduction. This is done by on-line calculation of the correlation between FES stimulation provided by the stabilizer and actual muscle movements. For example, the system may learn that applying specific voltage or other parameters of the electrical stimulation pattern, such as frequency, pulse duration, waveform etc results in a certain movement of the muscle (on average).

Over time, the system studies what stimulation should be given for a required response. Using this knowledge, the system knows what stimulation should be applied to the patient to oppose vibration. The method of the invention may therefore be used, without manual calibration, on different patients, in various electrodes placements and will improve its stabilization over time, and adapt to naturally occurring physiological changes of the stimulated muscle tissues.

It is noted that the methods of the present invention have many applications. The methods and systems of the invention can be adapted to a variety of body sites, and can be adapted to use a variety of sensing parameters both individually and in combination. The methods and systems can be adapted to work with more than one pair of antagonistic muscles.

As just one of many examples of applications of the invention, the methods and systems of the present invention may be used for counterbalancing fatigue. The system may be switched on and off according to the user's needs (e.g., feeling of fatigue). The methods and systems of the present invention may be used by military/police forces for combating fatigue and reducing tremors of shooters, so as to stabilize the shooter's hand and improve aim.

It will be appreciated by persons skilled in the art that the present invention is not limited by what has been particularly shown and described hereinabove. Rather the scope of the present invention includes both combinations and sub-combinations of the various features described hereinabove as well as variations and modifications which would occur to persons skilled in the art upon reading the specifications and which are not in the prior art

1. A method for tremor reduction comprising:
sensing tremor movements of at least one part of a body;
providing Functional Electrical Stimulation (FES) to a muscle;
generating a set of relationships of muscle response to the FES, called FES-muscle-response relationships; and
selecting and applying a new FES to the at least one part of the body in accordance with acquired knowledge of said FES-muscle-response relationships.

2. The method according to claim 1, wherein the new FES is selected so as to oppose said tremor movements.

3. (canceled)

4. The method according to claim 1, wherein the new FES is selected by on-line calculation of a correlation between FES stimulation and actual muscle movements.

5. The method according to claim 1, further comprising:
sensing further body tremor movements;
comparing the further body movements with previously stored FES-muscle-response relationships;
selecting the new FES in accordance with the previously stored FES-muscle-response relationships wherein the new FES is selected so as to oppose unwanted movement of the muscle; and
applying the new FES to oppose unwanted movement of the muscle.

6. The method according to claim 1, further comprising modifying the FES-muscle-response relationships in response to a change associated with the muscle being stimulated.

7. The method according to claim 1, wherein sensing the tremor movements comprises sensing an acceleration of at least one of a patient's limb and neck associated with the muscle by means of an accelerometer.

8. The method according to claim 1, comprising providing FES to a plurality of muscles, each muscle being activated by a separate FES circuit.

9. The method according to claim 8, wherein said FES circuits are adapted to generate pulses that are modulated by control signals that control contraction of the muscles.

10. The method according to claim 9, wherein said FES circuits are adapted to generate pulses that are modulated by two control signals, denoted U_{d}(n),D_{d}(n) respectively, wherein as an amplitude of U_{d}(n),D_{d}(n) increases, the corresponding muscle is affected to generate stronger contraction, and wherein the control signals are defined by:
$${C}_{d} ( n ) = - {I}_{d} ( n )$$
$${U}_{d} ( n ) = \{ \begin{matrix}{β}_{1} ( n ) {C}_{d} ( n ) & {C}_{d} ( n ) \geq 0 \\ 0 & {C}_{d} ( n ) < 0\end{matrix} \\ {D}_{d} ( n ) = \{ \begin{matrix}{β}_{2} ( n ) {C}_{d} ( n ) & {C}_{d} ( n ) \leq 0 \\ 0 & {C}_{d} ( n ) > 0\end{matrix}$$
and wherein selecting the new FES comprises:
defining positive and negative tremor energy as the tremor energy during N samples:
$${S}^{P} ( n ) = {\sum }_{n = 0}^{N - 1} {[ max \{ {C}_{d} ( n ) , 0 \} ]}^{2}$$
$${S}^{N} ( n ) = {\sum }_{n = 0}^{N - 1} {[ min \{ {C}_{d} ( n ) , 0 \} ]}^{2} ;$$
and adjusting values of the parameters β₁(n),β₂(n) to minimize both positive and negative tremor energy, according to:
$${β}_{1} ( n ) = {β}_{1} ( n - 1 ) - Δ \cdot \frac{{S}^{P} ( n - 1 ) - {S}^{P} ( n - 2 )}{{β}_{1} ( n - 1 ) - {β}_{1} ( n - 2 )}$$
$${β}_{2} ( n ) = {β}_{2} ( n - 1 ) - Δ \cdot \frac{{S}^{N} ( n - 1 ) - {S}^{N} ( n - 2 )}{{β}_{2} ( n - 1 ) - {β}_{2} ( n - 2 )}$$

11. (canceled)

12. Apparatus for tremor reduction comprising:
a sensor for sensing movements of at least one part of a body;
a stimulation/recording electrode unit for providing Functional Electrical Stimulation (FES) to a muscle; and
a processor for generating a set of relationships of muscle response to the FES, called FES-muscle-response relationships, and for selecting a new FES for application to the muscle in accordance with acquired knowledge of said FES-muscle-response relationships.

13. The apparatus according to claim 12, wherein said stimulation/recording electrode unit comprises:
a first layer comprising an adhesive and conductive interface layer adapted to directly interface with a skin surface and to provide electrical contact for conducting electrical energy to the skin surface for effecting muscle contraction and for detecting bio-potential signals indicative of neuro-muscular activation;
a second layer comprising a flexible conducting layer adapted to provide electrical contact between the first layer and a source of electromotive force (EMF) and to electrically connect the skin surface to a control unit for electrical stimulation of the skin surface; and
a third layer comprising an outer flexible layer that includes a source of electrical energy and serves as a protective covering layer, said third layer comprising a portal for installing therein said control unit.

14. (canceled)

15. The apparatus according to claim 12, wherein said sensor comprises an accelerometer.

16. The apparatus according to claim 12, wherein said sensor comprises an electromyographic activity detector.

17. The apparatus according to claim 12, wherein a control unit is disposed in said outer flexible layer, said control unit being adapted to regulate feedback-controlled stimulatory activity and to receive input information.

18. (canceled)

19. The apparatus according to claim 12, further comprising an alignment and electrode application unit on which said stimulation/recording electrode unit is mounted, said alignment and electrode application unit being adapted to mount said stimulation/recording electrode unit on at least one of a patient's limb and neck, wherein said alignment and electrode application unit restricts relative movement between said stimulation/recording electrode unit and a patient's limb or neck, respectively, along a predefined path.

20. (canceled)

21. The apparatus according to claim 19, wherein said stimulation/recording electrode unit comprises interface magnets and said alignment and electrode application unit comprises interface magnets corresponding to a position of said interface magnets of said stimulation/recording electrode unit but of opposing polarity, to prevent inverse placement of said stimulation/recording electrode unit on the patient's arm.

22. The apparatus according to claim 19, wherein a control unit is disposed in said outer flexible layer, said control unit being adapted to regulate feedback-controlled stimulatory activity and to receive input information, and wherein said stimulation/recording electrode unit comprises interface magnets and said control unit comprises interface magnets corresponding to a position of said interface magnets of said stimulation/recording electrode unit but of opposing polarity, to prevent inverse placement of said control unit with respect to said stimulation/recording electrode unit.

23. The apparatus according to claim 19, wherein said alignment and electrode application unit comprises a pathway for insertion of a patient's limb therethrough, said stimulation/recording electrode unit being positioned near said pathway such that when the patient's limb passes through the pathway the stimulation/recording electrode unit becomes mounted on the patient's limb, and wherein the patient's limb is forced to pass through said pathway in a predefined orientation with respect to said stimulation/recording electrode unit.

24-25. (canceled)

26. The apparatus according to claim 12, wherein said stimulation/recording electrode unit comprises a filter for filtering around a tremor frequency to ignore slow movements and high frequency noise associated with the muscle movements that were sensed.

27-28. (canceled)
